(19) United States
(12) Patent Application Publication
(10) Pub. No.: US 2026/0315132 A1
(43) Pub. Date: Oct. 15, 2026

(54) ULTRASOUND-GUIDED HEAT STEERING MECHANISM AND TISSUE PROPERTY SENSING FOR ENHANCED CATHETERS PRECISION FOR ABLATION

(71) Applicant: THE UAB RESEARCH FOUNDATION, Birmingham, AL (US)

(72) Inventors: Seyedabdollah Mirbozorgi, Irondale, AL (US); Venkata Suvarchala Priyanka Jamjhyam, Birmingham, AL (US)

(21) Appl. No.: 19/552,997

(22) Filed: Feb. 27, 2026

Related U.S. Application Data
(60) Provisional application No. 63/765,237, filed on Feb. 28, 2025.

(51) Int. Cl. A61B 18/08; A61B 17/00; A61B 18/00; A61B 18/02; A61B 34/20; A61M 25/01

(52) U.S. Cl. CPC A61B 18/082; A61B 34/20; A61B 2017/00292; A61B 2018/00577; A61B 2018/00642; A61B 2018/00714; A61B 2018/00761; A61B 2018/00791; A61B 2018/00994; A61B 2018/0212; A61B 2018/087; A61B 2034/2051; A61M 25/0127

(57) ABSTRACT

One method, among others, of the present disclosure involves positioning a catheter in a portion of tissue during an ablation, where the catheter comprises a heat source and a control unit for controlling individual components of the catheter; and generating, by the heat source, a transfer of heat, wherein the heat source alternates heating and freezing cycles at different frequencies to achieve a near zero-temperature gradient at a tumor boundary. Other methods and systems are also provided.

### CROSS-REFERENCE TO RELATED APPLICATION

This application claims priority to co-pending U.S. provisional application entitled, “Ultrasound-Guided Heart Steering Mechanism and Tissue Property Sensing for Enhanced Catheters Precision for Ablation,” having application No. 63/765,237, filed Feb. 28, 2025, which is entirely incorporated herein by reference.

### STATEMENT REGARDING FEDERALLY SPONSORED RESEARCH OR DEVELOPMENT

This invention was made with government support under 2338890 awarded by the National Science Foundation. The government has certain rights in the invention.

### BACKGROUND

Thermal tumor ablation is an invasive procedure that uses heat to destroy tumor cells. It is commonly used to treat brain, liver, kidney, bone, and lung tumors. Above 10 million cancer cases are projected to be diagnosed each year globally by 2030, which can be treated via thermal ablation. Current ablation technologies (microwave, radio frequency, laser, and focused ultrasound), have significant limitations that result in the heat generated not being confined to the target area, putting adjacent tissues at risk of being damaged. Especially for brain tumors, it is essential that the procedure is highly accurate to minimize damage to healthy brain cells, which can lead to significant consequences (nerve damage, losing abilities), and avoid incomplete ablation that can cause recurrence. The brain tumor recurrence rate is over 25% and up to 62.5% for incomplete ablations. The heat increases almost symmetrically in all directions, while most tumors have asymmetrical shapes with different sizes. Although researchers are actively working on developing approaches for creating asymmetric coagulation ablation zones, which mainly offer solutions with limited capabilities, the currently available commercialized technologies do not have the capability to direct heat in specific direction.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 and FIG. 2 show an exemplary Tumor Precision Ablation (TPA) catheter system in accordance with various embodiments of the present disclosure.

FIG. 3 shows a comparison of ablation thermal boundary via conventional ablation and a Tumor Precision Ablation in accordance with the present disclosure.

FIG. 4 shows temperature variations as a function of time and distance radius for constant and sinusoidal heat flux models (MATLAB) of the disclosed system in accordance with the present disclosure.

FIG. 5 shows a block diagram of various components of an exemplary TPA catheter system, in accordance with various embodiments of the present disclosure.

FIG. 6 demonstrates a working principle of a Peltier module, in accordance with the present disclosure.

FIG. 7 shows a design overview of an exemplary Peltier-based catheter PBC in COMSOL Multiphysics showing realistic setup of Peltier module in biological tissue model of fat, in accordance with the present disclosure.

FIG. 8 shows COMSOL simulation results showing temperature contours sliced along the Y-Z plane for two current excitation modes in accordance with the present disclosure.

FIG. 9 shows COMSOL simulation results showing temperature contours sliced along the Y-Z plane for two current excitation modes in accordance with the present disclosure.

FIG. 10 shows the temperature variation as a function of distance (upper half: 0-20 mm) for a constant DC input current (C) of 1.5 A (0 Hz) and an alternating current (AC) input of 1.5 A with a frequency of 1/120 Hz (at/before 60 s, 120 s, 180 s, 240 s, and 300 s), in accordance with the present disclosure.

FIG. 11 shows the differential temperature variation as a function of distance (upper half: 0-20 mm) for a constant DC input (0 Hz) and alternating current inputs with frequencies of 1/240 Hz, 1/120 Hz, and 1/20 Hz, all at an input current of 1.5 A after 300 s, in accordance with the present disclosure.

FIG. 12 shows the distance of heat penetration as a function of input frequency for a Peltier module driven with a constant DC input (0 Hz) and alternating current inputs at different frequencies after 300 s, in accordance with the present disclosure.

FIG. 13 shows the distance of heat traveled as a function of time for a constant DC input and alternating current inputs with frequencies of 1/120 Hz, 1/60 Hz, 1/40 Hz, and 1/20 Hz, in accordance with the present disclosure.

FIGS. 14A-14D illustrates (A) the heat directing principle, (B) hardware integration of ultrasound arrays with the heat source, and (C) an alternative solution using two tilted piezo elements as the heat source, and (D) a piezo array model and beamforming results for an exemplary TPA catheter, in accordance with various embodiments of the present disclosure.

FIG. 15 shows a conceptual illustration of an exemplary catheter-based fractal UWB antenna for evaluating the brain tumor ablation depth and location, in accordance with various embodiments of the present disclosure.

FIG. 16 shows an illustration of an exemplary UWB fractal sensor in flat view and its associated low-mid-high frequency ranges to the fractal regions, in accordance with various embodiments of the present disclosure.

FIG. 17 shows the 3D view and front view cross section of an exemplary TPA catheter with UWB sensing antenna, including the dimensions details, in accordance with various embodiments of the present disclosure.

FIG. 18 shows an HFSS model of a multilayer human head with an ablated tissue region embedded in the brain layer, in accordance with the present disclosure.

FIG. 19 shows side and front views of the tissue model and an exemplary catheter, showing the details of sensing antenna and its isolation layers, wrapped around the catheter body, in accordance with various embodiments of the present disclosure.

FIG. 20 shows the S11 simulation result based on burn tissue locations changing in accordance with the present disclosure.

FIG. 21 shows the simulation result of S11 in different Burn Tissue size variations in accordance with the present disclosure.

FIG. 22 shows the simulation result of S11 in different Burn Tissue size variations in accordance with the present disclosure.

FIG. 23 shows a Poynting vector simulation of a fractal antenna at different frequencies, showing the dependency of wave propagation location to the frequency, in accordance with various embodiments of the present disclosure.

FIG. 24 shows an SAR calculation of an exemplary UWB sensor to evaluate the safety of using the sensor for the healthy brain cells, in accordance with various embodiments of the present disclosure.

FIG. 25 illustrates a summary of the assembly and integration process of an exemplary TPA catheter in accordance with various embodiments of the present disclosure.

FIG. 26 shows a COMSOL model and heat transfer simulation results for the disclosed system in accordance with various embodiments of the present disclosure.

FIG. 27 shows a COMSOL model and max or min achieved temperature as function of radius for the disclosed system in accordance with various embodiments of the present disclosure.

FIG. 28A shows measured temperature versus radius for a test setup of the disclosed system in accordance with various embodiments of the present disclosure.

FIG. 28B shows measured heat penetration radius vs time (room temperature: 295K) for a test setup of the disclosed system in accordance with various embodiments of the present disclosure.

FIG. 29 depicts an evaluation setup and test block diagram for the TPA catheter system in accordance with various embodiments of the present disclosure.

FIGS. 30A-30B show measured results of a feasibility study of directing heat with ultrasound waves; confirming the propagation of heat towards ultrasound direction (left to right), in accordance with the present disclosure.

FIG. 31A shows an HFSS model of an exemplary Ultra-Wideband (UWB) sensor along with S11 versus frequency simulation results, in accordance with the present disclosure.

FIG. 31B shows measured S11 results for a UWB sensor prototype, in accordance with the present disclosure.

### DETAILED DESCRIPTION

The present disclosure addresses the unmet demand for precise, reliable, and personalized tumor ablation technologies by introducing innovative discoveries and interventions in physics to control heat penetration depth, direct the heat in a desired direction for asymmetrical tumor shapes, and sense ablation stage/depth. Together these capabilities can enable closed-loop, real-time precision control of tumor ablation. In accordance with systems and methods of the present disclosure, periodically heating and freezing a tumor (with frequency f) limits the ablative heat penetration to a specific depth, while the intervention of heat transfer and ultrasound waves propagation results in directing heat at ultrasound wave direction, and the application of Ultra-Wideband (UWB) sensors acts to detect the depth of ablation.

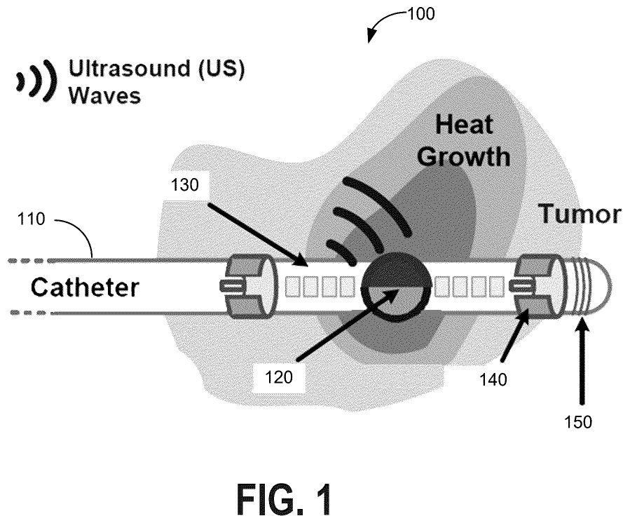

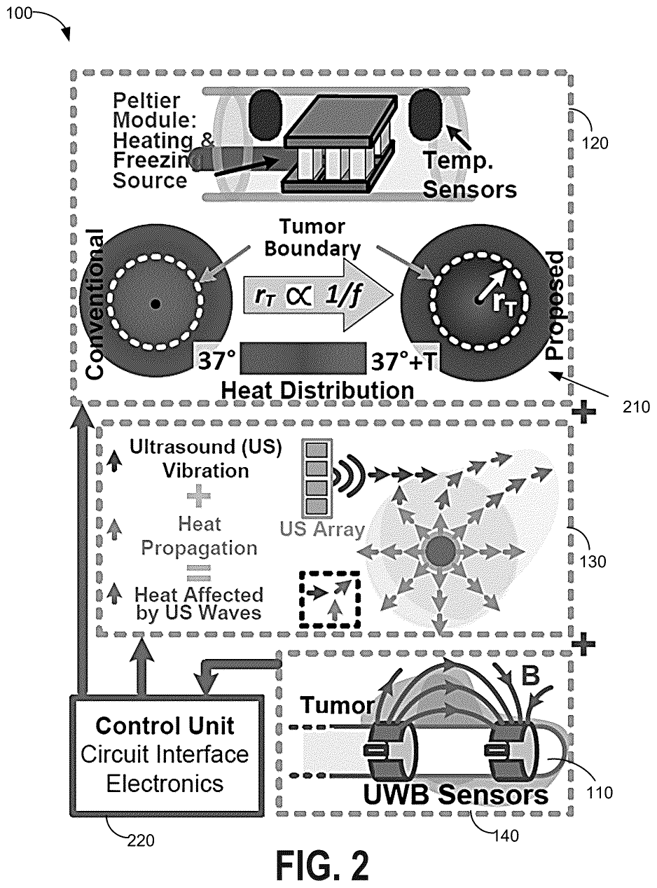

Accordingly, systems and methods of the present disclosure leverage electronics to control heat propagation in tissue layers via a novel multimodal Tumor Precision Ablation (TPA) catheter that combines a plurality of innovative technologies, as shown in FIG. 1. Here, an exemplary TPA catheter system 100 comprises a TPA catheter apparatus 110 having a heat source 120, an ultrasound (US) transducer array 130 to direct the heat transfer from the heat source 120, a UWB sensor (or UWB sensor array) 140 to detect changes in tumor properties during ablation, and a magnetic resonance imaging (MRI) coil 150 for tracking. Accordingly, in FIG. 1, the figure illustrates a tumor undergoing an ablation procedure with the TPA catheter 110. In FIG. 2, an exemplary TPA catheter system 100 is shown to further include a control unit 220 for controlling the individual components 120-150 of the TPA catheter 110.

In various embodiments, the heat source 120 for the exemplary TPA catheter 110 includes one or more Peltier modules that produce heating and freezing effects and may further include temperature sensors for detecting the temperature output by the catheter 110. Within the field of tumor ablation, an exemplary TPA catheter 110 is designed to (1) alternate heating and freezing cycles at different frequencies to achieve a near zero-temperature gradient at the tumor boundary (via the heat source 120), (2) direct heat in desired directions using ultrasound waves (a novel intervention) (via the US transducer array 130), and (3) detect changes in tumor properties during ablation (via the UWB sensor 140). Accordingly, in various embodiments, an exemplary TPA catheter 110 is equipped with a closed-loop control mechanism, enabling complete control over the propagation of heat in tissue layers and detection of the ablation progress by the control unit 220.

As mentioned above, an exemplary TPA catheter 110 is designed to alternate heating and freezing cycles at different frequencies to achieve a near zero-temperature gradient at a tumor boundary. To do so, heating and freezing phases are alternated at different frequencies to set (to a clinically defined radius r_{T}) the heat penetration depth with a near zero-temperature gradient at the boundary of the tumor, as shown in FIG. 2 by the disclosed heat distribution technique 210. In various embodiments, a mm-sized Peltier module 120 is incorporated in the TPA catheter 110 to generate the heating and freezing phases and apply different pulse patterns to control the heat penetration depth to investigate the correlation between the ablation radius and pulse patterns.

To effectively direct heat in a desired direction, the exemplary TPA catheter system 100 combines the principles of heat transfer and ultrasound wave propagation, which is grounded in the Phonon principle, which involves the transmission of energy through waves and vibrations, as well as the modulation of thermal conductivity (enhancement) achievable through the propagation of ultrasound waves in tissues. Accordingly, in various embodiments, an exemplary TPA catheter 110 enlists ultrasound transducer array(s) 130 to direct (by beamforming) the Peltier module 120 heat toward a desired direction using beamformed ultrasound waves.

In accordance with the present disclosure, changes in tissue dielectric properties at the ablation zone can be captured by implementing UWB sensors 140 at the tip of the catheter 100. In various embodiments, this real-time detection mechanism can enable a closed-loop control mechanism on ablation states.

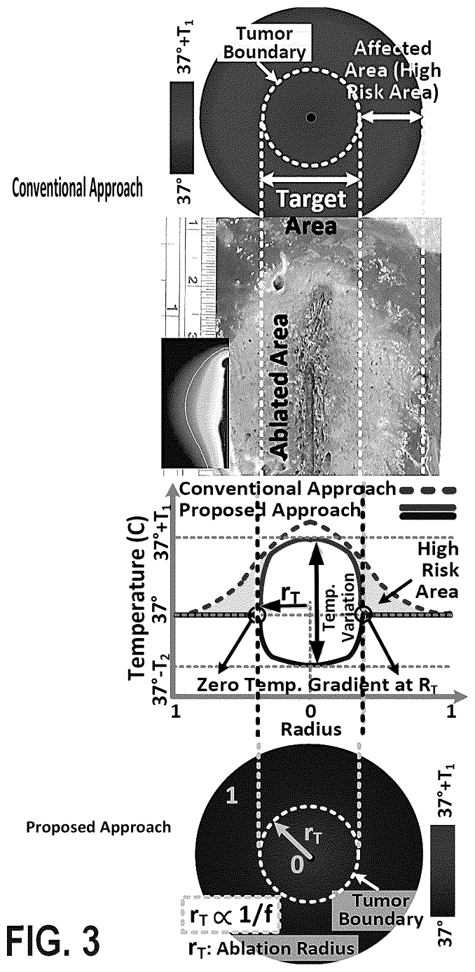

As a general overview, ablation is an invasive treatment method used for various applications, such as eliminating cancerous tumors in different organs, treating atrial fibrillation and ventricular tachycardia. Ablation techniques can be divided into nonthermal ablation (chemical ablation and percutaneous irreversible electroporation, IRE) and thermal ablation. Thermal ablation can involve heating (Thermal Ablation) or freezing (Cryoablation) to destroy tumor cells. Different techniques, including Radiofrequency ablation (RFA), Microwave ablation (MWA), High-Intensity Focused Ultrasound (HIFU) ablation, and laser interstitial thermal therapy (LITT), are used for thermal ablation. LITT has also emerged as an effective treatment option for mesial temporal lobe epilepsy (MTLE) associated with certain lesions. Cryoablation catheters use freezing to kill tumor cells by forming intracellular ice crystals. Dual thermal ablation, combining heat exposure and freezing, has been developed to enhance therapeutic outcomes. However, its application is focused on improving the kill level of cancer cells rather than controlling the ablation thermal boundary. Ablation treatment is typically suitable for tumors smaller than 3 cm and can pose challenges when targeting brain tumors or tumors near critical structures. Limitations of current ablation technologies include uncontrollable ablation boundaries resulting in heat affecting adjacent tissues, incomplete tumor cell destruction due to irregular tumor shapes leading to high recurrence rates, and the lack of a reliable feedback mechanism for monitoring the volume of ablated tissues during the ablation procedure. FIG. 3 illustrates the issue with current technologies (“conventional approach”), which pose a risk of damaging healthy tissues adjacent to the tumor. In comparison, the disclosed approach (“proposed approach”) of systems and methods of the present disclosure involves alternating the heating and freezing phases. This allows for the precise setting of the heat penetration depth within the therapist-defined radius (r_{T}), maintaining a near-zero temperature gradient at the tumor boundary.

Treating recurrent metastatic tumors and gliomas (malignant tumors of glial tissue) in the brain presents a significant challenge. Preservation of healthy brain cells and blood vessels adjacent to the tumor is crucial in any brain treatment. Leaving some cancer cells near the tumor edges is necessary to avoid damaging healthy brain cells, but it often leads to recurrences. Almost all brain tumors recur after treatment. Thermal ablation on the brain is approached with caution due to the difficulty in controlling the depth of heating and the associated risk of neurological deficits. The irregular shape of tumors further complicates treatment.

Delayed feedback on cell viability is a concern since patients need to wait for a week to obtain MRI or computed tomography (CT) scans for assessment. Periodic follow-up MRIs (every 1 to 6 months) are required to monitor tumor recurrence. Sensing temperature accurately becomes challenging due to the non-uniform thermal landscape caused by factors like larger blood vessels acting as heat sinks.

The exemplary TPA catheter system 100 of the present disclosure may accelerate the process by subjecting cells to alternating heat flux, with cell death potentially being proportional to the temperature gradient. Additionally, cooling through the TPA catheter 110 can potentially mitigate swelling issues, and employing various frequencies for switching between heating and freezing phases can control the boundary of the ablation area and spare healthy tissues. In various embodiments, an exemplary TPA catheter system 100 incorporates a small MRI coil 150 on the catheter 110 to track its tip during MRI-guided implantation without interfering with tumor images, while thermal energy is directed to specific locations within the irregular shapes of tumors using beamformed ultrasound waves. As such, an implantable Peltier-based TPA catheter device with alternating thermal cycles, in accordance with the present disclosure, may provide a promising solution for more effective tumor treatment.

In accordance with the present disclosure, alternating heating and freezing cycles are applied by the TPA catheter system 100 to control and limit the ablation thermal boundary, minimizing the temperature rise in adjacent tissue layers compared to conventional thermal ablation techniques. Heat transfer will be assumed to be conductive. For the scenario considered here, it can be modeled analytically using the heat equation (1) (below) in spherical coordinates.

$$\frac{\partial T}{\partial t} = \frac{α}{{r}^{2}} \frac{\partial }{\partial r} ( {r}^{2} \frac{\partial T}{\partial r} ) \qquad ( 1 )$$

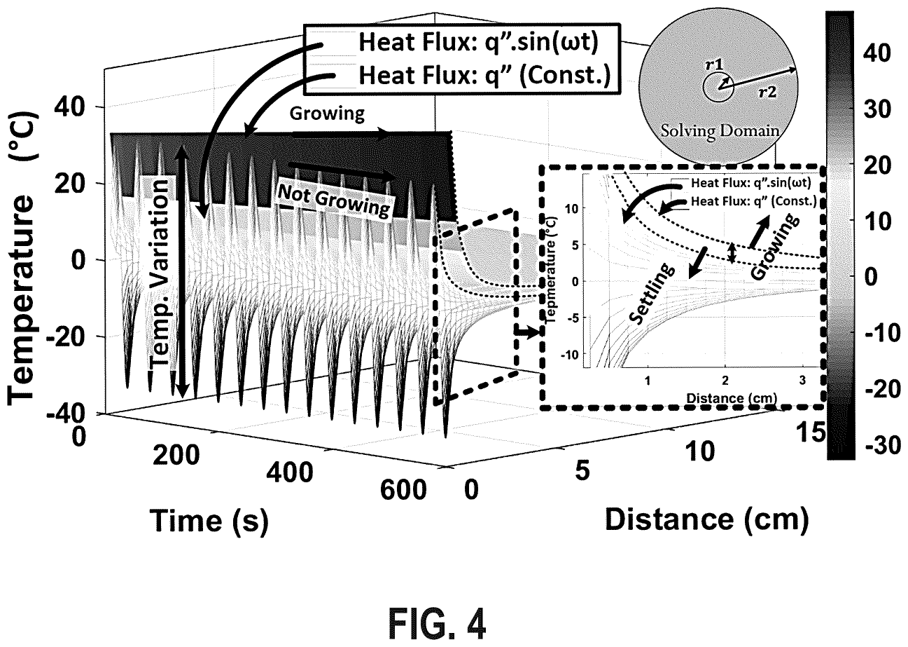

The boundary conditions used are ∂T/∂r|_(r=r₂)=0 and ∂T/∂r|_(r=r₁)=(−q″)/k where T is the temperature, t is the time, r is the radius, α=k/(ρCₚ is the thermal diffusivity, k is the thermal conductivity of the medium (W/mK), Cₚ is the specific heat capacity, ρ is the density, and q″ is the constant heat flux (W/m²). The boundary conditions used are ∂T/∂r|_(r=r₂)=0) and ∂T/∂r|_(r=r₁)=(−q″)/k sin (ωt) to solve Eq. (1), where w is the angular frequency (ω=2πf) and f is the frequency of heating and freezing. MATLAB was utilized to plot the solutions of Eq. (1) for both conditions, and the results are presented in FIG. 4, which is an initial and ideal model of the disclosed system. The figure illustrates the temperature variations over time and distance from r₁ (radius of the Peltier module model) to r₂ (radius of the solution domain) for constant heat flux in conventional heat transfer and sinusoidal heat flux in the exemplary approach. It is evident that with constant heat flux, the penetration depth continues to increase, and the temperature does not return to the relative human body temperature. In contrast, with the disclosed alternating heat flux, the temperature settles back to zero, indicating that the tissue layers have not experienced significant temperature changes.

As discussed, in various embodiments, one or more Peltier modules are utilized as the heat source 120 of the TPA catheter 110, which operates based on the thermoelectric effect. The junction connecting two thermoelectric materials emits or absorbs heat per unit time to balance the difference in chemical potential and create a temperature difference. These thermoelectric materials have low thermal conductivity and high electrical conductivity, typically semiconductor types N & P. The direction of the electrical current in a Peltier module determines the heated and cooled surfaces. In an exemplary non-limiting prototype, a small Peltier module (2.5×2.5×2.5 mm3) with a power capacity of ~1 W is employed (00801-9B30-10RU3).

In general, the Peltier module has two surfaces (junctions), in which it generates heat on one surface and cools the other, simultaneously. Accordingly, the Peltier module is configured to absorb heat from one surface and transfer the heat to the opposite surface using input electrical energy. In so doing, the generated thermal energy heats the tissue, facilitating the required chemical reactions for converting tumor tissue into burned tissue (ablation). In various embodiments, separation of the freezing and heating surfaces of a Peltier module enable pure heating or pure freezing at the tip of the catheter 110. In various embodiments, the system design includes the heat sink and overall heat transfer processes to achieve efficient freezing and heating effects.

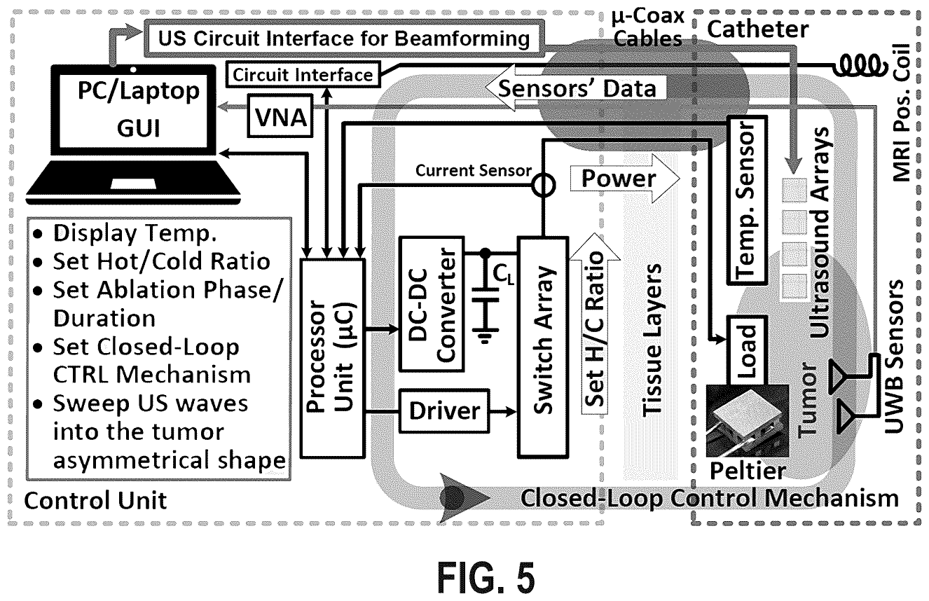

In various embodiments, an exemplary TPA catheter system 100 incorporates interface control circuits and a processor unit as part of the control unit 220 to control the Pelter modules 120 and change heating and freezing phases, adjust power levels, and incorporate temperature sensors, as shown in FIG. 5. Additionally, as shown in FIG. 5, the closed-loop control mechanism supplies sensor data from the catheter 110 to the control unit 220 which in turn provides signals for setting ablation phase/duration parameters, transmitting ultrasound waves into tumor tissue, etc. In various embodiments, a graphical user interface (GUI) of the control unit 220 is provided to control the catheter system 100.

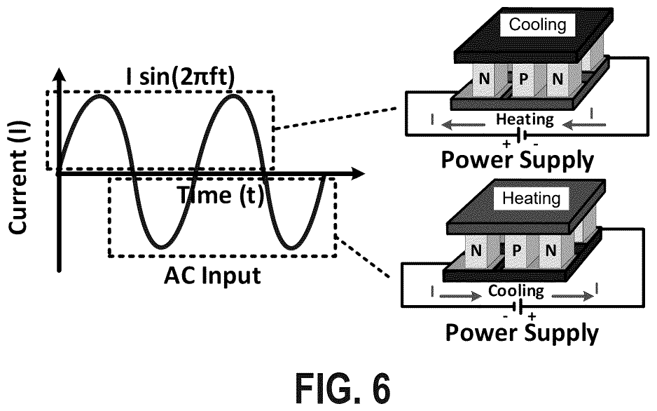

In various embodiments, the disclosed TPA catheter is specifically designed for insertion into brain tissue (which exhibits similar thermal properties to fat material), facilitating precise tumor ablation. FIG. 6 outlines the working principle of a Peltier module of the TPA catheter, where changing the current input direction leads to changing the heating and cooling plates. The Peltier module operates based on the Peltier effect, where heat is absorbed or released at the junctions of dissimilar semiconductors when an electric current flows through them. Alternating N-type and P-type Bismuth Telluride legs form these junctions, enabling controlled heating or cooling depending on current direction. This localized thermal generation is used to generate the thermal energy necessary for heating tissue, thereby facilitating chemical reactions required for converting tumor tissue into ablated (burned) tissue.

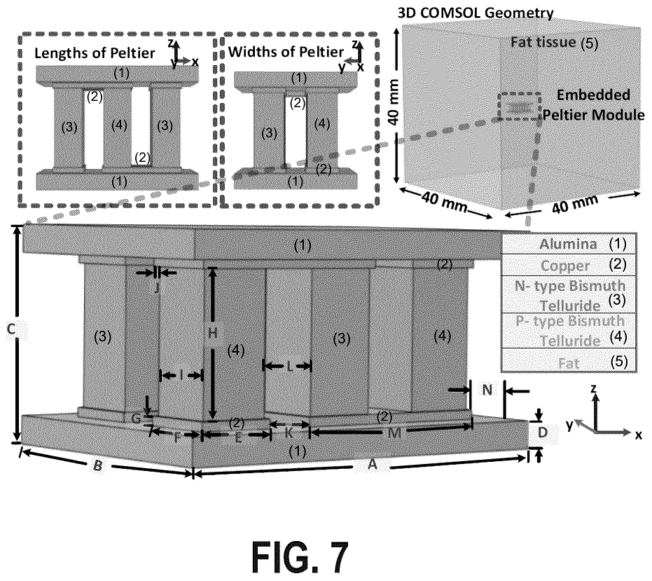

To evaluate the effectiveness of an exemplary TPA catheter, a computational simulation was performed using COMSOL Multiphysics software, incorporating realistic properties of fat tissue which represents the brain tissue. Comprehensive design details of the modeled Peltier-based catheter (PBC) is presented in FIG. 7, including its integration within realistic fat tissue. A three-dimensional PBC model was developed in COMSOL to evaluate localized thermal ablation. The Peltier module may be formed of alternating N-type and P-type Bismuth Telluride thermoelectric legs sandwiched between copper conductors and alumina substrates for electrical insulation and mechanical support. In a non-limiting design, the compact module has dimensions of 5.2 mm (width), 3.6 mm (depth), and 2.3 mm (height). It includes six thermoelectric legs, each 1.5 mm in height, with an inter-leg spacing of 0.7 mm. The module is directly embedded at the center of a realistic fat/brain tissue domain measuring 40 mm×40 mm×40 mm. The design overview and geometric parameters are defined in FIG. 7 and summarized in Table I (below).

TABLE I
Peltier module parameters (mm)

| | |
|---|---|
| A | 5.2 |
| B | 3.6 |
| C | 2.3 |
| D | 0.3 |
| E | 1.0 |
| F | 1.0 |
| G | 0.1 |
| H | 1.5 |
| I | 0.9 |
| J | 0.1 |
| K | 0.6 |
| L | 0.7 |
| M | 2.6 |
| N | 0.5 |

For modeling the TPA catheter device, COMSOL Multiphysics was employed with the Heat Transfer in Solids and Fluids, Electric Currents, Thermoelectric Effect, and Electromagnetic Heating interfaces. The Heat Transfer in Solids and Fluids interface was utilized to simulate heat transfer with the Peltier module and to analyze temperature distribution within the fat/brain media. The Electric Currents interface modeled the input current applied to the Peltier module, while the Thermoelectric Effect and Electromagnetic Heating interfaces were critical for accurately simulating the heat generation from electric currents in the Peltier module. A physics-controlled finer mesh was applied throughout geometry to enhance the accuracy of numerical analysis. Initial temperature conditions for the Peltier module and fat/brain tissue were set at 37° C. (human body temperature).

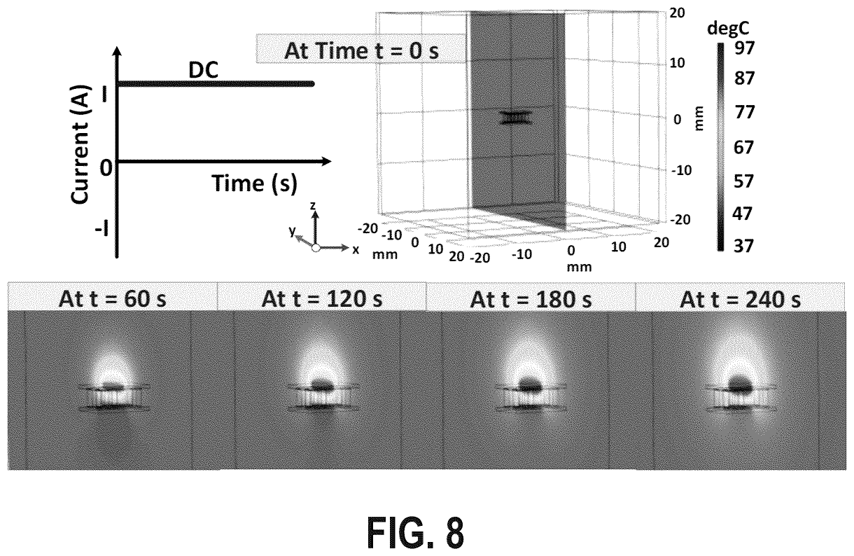

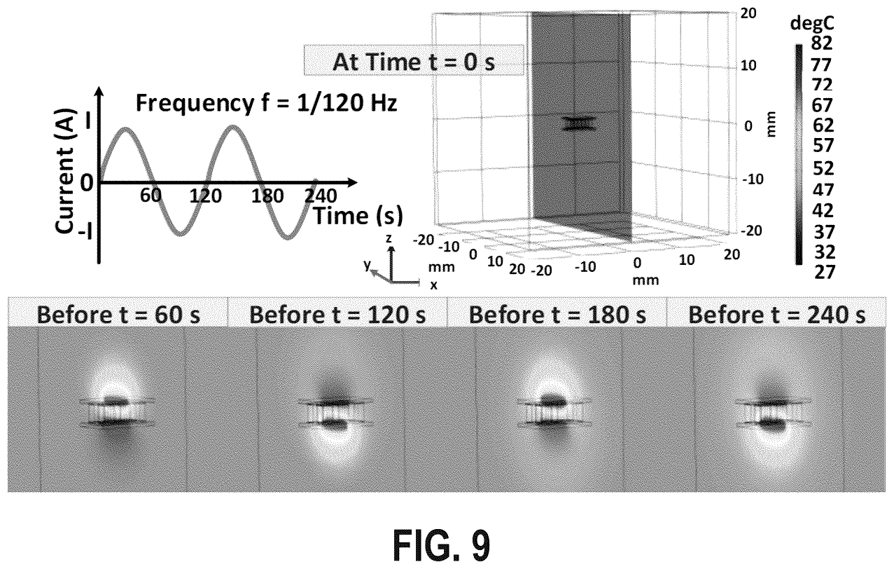

The Peltier-based catheter (PBC) system shown in FIG. 7 was modeled and analyzed using a time-dependent simulation in COMSOL Multiphysics over a total duration of 300 seconds. The resulting temperature distributions were evaluated as a function of spatial distance and thermal response for different input current frequencies. FIG. 8 illustrates the temperature contours for a constant DC input current of 1.5 A (0 Hz). As time progresses, the heat generated by the Peltier module continuously accumulates within the surrounding tissue, leading to a rapid and uncontrolled expansion of the thermal region, which is observable in FIG. 8. FIG. 9 presents the temperature contours for an alternating current (AC) input of 1.5 A with a frequency of 1/120 Hz, simulated for the two complete cycles. In this case, the input current alternates every 60 seconds following Eq. (2), resulting in periodic heating and cooling cycles.

I(t)=I sin(2πft) (2)

where I is the input current magnitude, f is the input frequency, and t is the time. Due to this temporal alternation, the heat distribution within the tissue remains spatially confined within the confined range. These results clearly demonstrate that the disclosed alternating AC input approach effectively restricts rapid heat propagation compared to the constant DC input case.

When a constant positive current is applied to the Peltier module, heat is generated primarily on the upper side of the device, therefore, the analysis focuses on the upper half of the tissue domain (0-20 mm). In contrast, alternating current inputs, heating and cooling occur periodically. To maintain consistency and improve clarity in comparison, only the upper half of the domain was considered in all cases. Accordingly, a Y-Z plane was introduced, and all temperature profiles were extracted along this plane. Since temperature variations under 4° C. do not cause significant effects on the human body tissue, a temperature threshold of 3.5° C. was selected.

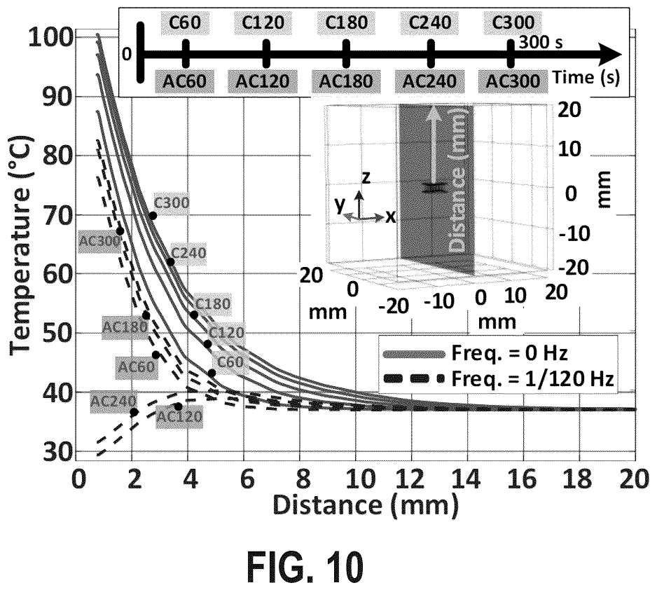

FIG. 10 compares the COMSOL simulation results for a constant DC input and an alternating input with a frequency of 1/120 Hz. For the constant DC input, the heat distribution increases rapidly with time. Although the spatial spread appears reduced at 240 seconds, extending the simulation duration would result in further heat propagation. In contrast, the alternating input confines the thermal distribution due to periodic heating and cooling cycles. As shown, the heat penetration is limited when alternating heating and cooling phases and by continuing the simulation, it will change insignificantly.

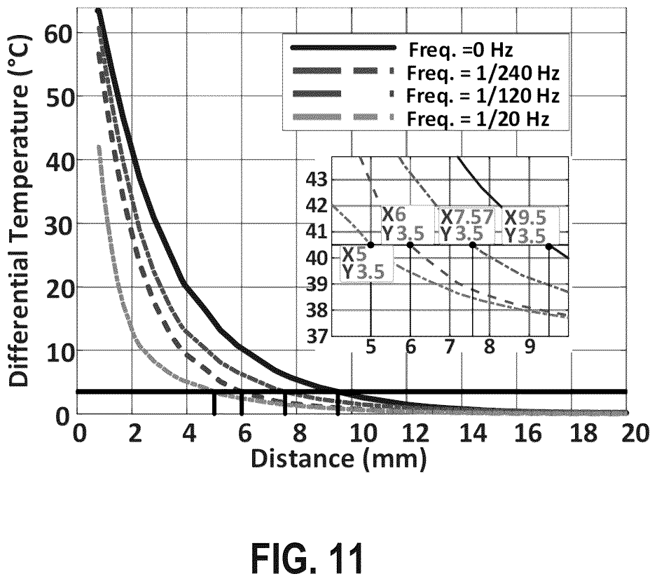

From FIG. 11, it is observed that the heat distribution is inversely related to the input frequency, i.e., as the input frequency increases, the thermal gradient decreases. Based on the selected temperature threshold of 3.5° C., a reference line at 40.5° C. was used to determine the depth of heat penetration for different input frequencies. For a frequency of 0 Hz, the heat penetrates up to 9.5 mm. At frequencies of 1/240 Hz, 1/120 Hz, and 1/20 Hz, the corresponding penetration depths are 7.57 mm, 6 mm, and 5 mm, respectively. These results demonstrate that higher-frequency alternating inputs effectively minimize temperature gradients, thereby reducing thermal impact on surrounding healthy tissue.

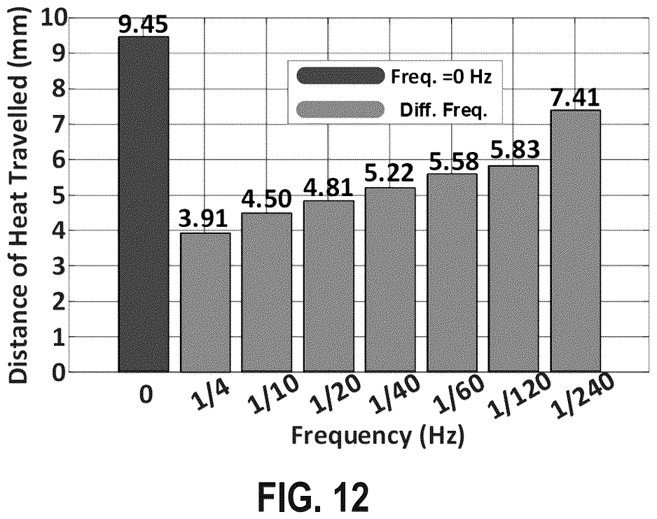

For the constant DC input (0 Hz), the heat penetration distance is the highest, which is 9.45 mm, indicating continuous and uncontrolled heat accumulation within the tissue. In contrast, alternating current inputs significantly reduce the spatial extent of heat propagation, observed from FIG. 12. This behavior is attributed to the periodic reversal of heating and cooling cycles, which limits sustained heat buildup within the tissue.

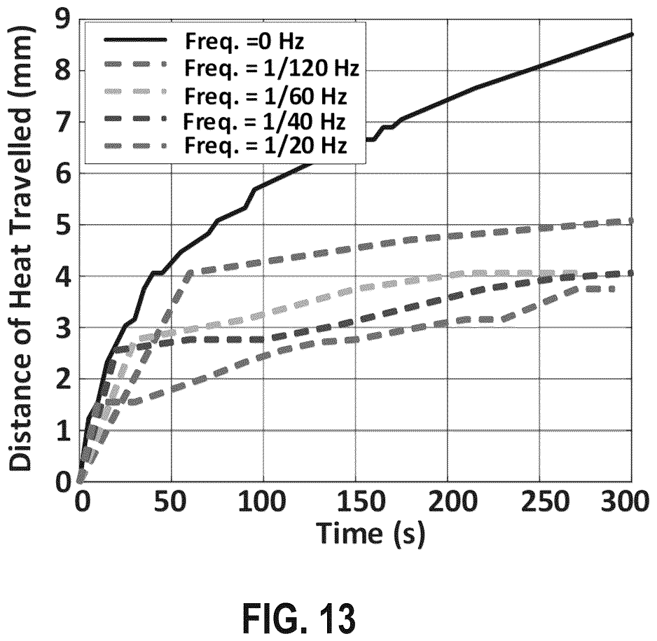

FIG. 13 illustrates the time-dependent variation of heat penetration distance for constant DC and alternating input frequencies. For the constant DC input, the thermal distribution increases continuously with time, indicating sustained heat accumulation. In contrast, for alternating input frequencies, the heat penetration distance reaches saturation after a certain period. This behavior highlights the critical role of temporal current modulation in regulating heat propagation. The periodic alternation between heating and cooling cycles suppresses excessive thermal buildup, resulting in a reduced temperature gradient and minimizing the thermal impact on surrounding healthy tissue.

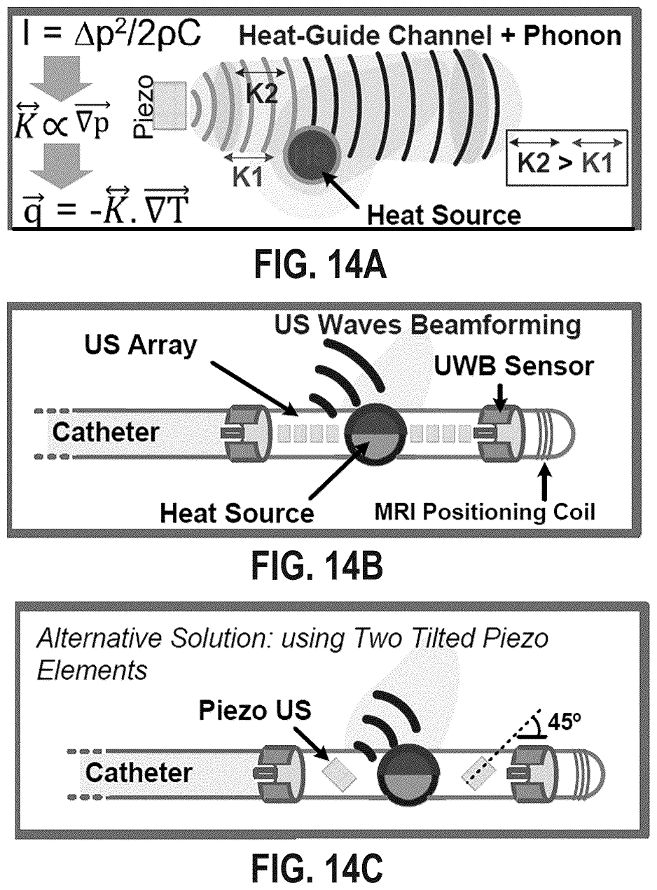

In accordance with systems and methods of the present disclosure, ultrasound waves transmitted by the TPA catheter 110 induce transient anisotropic behavior in a tumor tissue medium, that enhance thermal conductivity along their direction of propagation. This phenomenon creates a heat-guiding channel, that facilitates the propagation of heat with a higher heat flux within that channel, as illustrated in FIG. 14A. In accordance with the present disclosure, heat propagation and ultrasound wave transmission can be combined, as they both involve molecular vibration. Heat transfer relies on energy carriers like phonons, electrons, fluid particles, and photons. One focus of the present disclosure is on the phonon phenomenon, which refers to lattice vibration. Heat flux (\vec{q}) depends on the thermal conductivity ( [symbol] ) and the temperature gradient (\sqrt{∇T}) according to Fourier's law of conduction: \vec{q}=− [symbol] ·\vec{∇T} where \vec{∇T} represents the temperature derivative with respect to space (dT/ds). Ultrasound transmission's sound intensity (I) depends on the square of the differential sound pressure (Δp), sound speed (C), and density (ρ) of the medium (I=Δp²/2ρC). Ultrasound pressure increases medium density in the direction of sound propagation, which is linearly related to thermal conductivity. Thus, an increase in thermal conductivity ( [symbol] ) results in increased heat transfer. If this increase is directional (aligned with ultrasound waves), heat transfer becomes directional. Controlling ultrasound propagation direction is possible using an array of ultrasound transducers and beamforming.

In various embodiments, an exemplary ultrasound transducer array comprises a 1D array of ultrasound transducers. In certain non-limiting implementations, an optimal number of transducer piezo elements may be 4 to 8. Hardware components including amplifier circuits, signal generators, and power supply units, may be integrated to drive the transducer array. FIG. 14B illustrates an exemplary assembly approach of two ultrasound arrays 130 adjacent to the heat source 120 in the catheter 110, enabling heat directing forward and backward in different directions. The catheter 110 can be rotated during the ablation procedure to cover the entire tumor. FIG. 14D shows the simulation results of an ultrasound array model of FIG. 14B demonstrating its beamforming ability. In various embodiments, the graphical user interface (GUI) of the control unit 220 is configured to control the ultrasound transducer array 130 for beamforming and monitoring. Alternatively, FIG. 14C demonstrates a catheter design where only two ultrasound transducer elements are used, tilted ±45° (fixed, but can be manually adjusted). This approach simplifies the assembly process, eliminates the need for beamforming, and requires less space, while requiring catheter movement to sweep the tumor area. Tilting the ultrasonic transducer element allows for the angle of attack to be modified based on the target tumor.

In accordance with systems and methods of the present disclosure, UWB technology is deployed to detect tissue properties, including dielectric measurements, by measuring the absorption and reflection of UWB signals in different body tissues. In particular, systems and methods of the present disclosure can detect the volume of ablated tissue by comparing it with unablated tissue, enabling a closed-loop control mechanism for the ablation procedure.

Ultra-wideband (UWB) sensors/antennas 140 operating in the frequency range of 3 GHz to 12 GHz are versatile sensors used in various applications such as glucose monitoring, microwave imaging, breast cancer detection, and short-range biomedical communication. These antennas offer advantages in terms of cost, design simplicity, and flexibility. UWB microwave technology is widely used for early-stage breast tumor detection by wearable sensing arrays.

UWB sensors 140 propagate and receive electromagnetic waves, and the efficacy of radiation and penetration depends on the surrounding materials in their near field. The reflection and transmission coefficients, measured by the scattering parameters S11 and S21, provide information about the surrounding material properties (in 3D volume of the near field of the antenna), which will be influenced by the ablation procedure.

In various embodiments, the UWB sensing antenna 140 will be designed using HFSS software, with a cylindrical shape wrapped around the catheter tip. As a non-limiting example, the UWB sensing antenna (sensor) 140 may detect changes in the surrounding tissue within a radius of up to 2 cm. The design of the UWB sensor 140 may be optimized to maximize sensitivity in the UWB frequency range. In various embodiments, two UWB sensors 140 can be used to evaluate tissue changes between sensors by measuring the transmission coefficient (S21).

In various embodiments, multiple UWB sensors comprise flexible printed circuit boards (PCBs) (<0.3 mm thickness) covered with biocompatible isolation layers. In various embodiments, these sensors are integrated around the catheter (<2 mm radius) and a μ-coaxial cable connects the UWB sensor to a Vector Network Analyzer (VNA) (see FIG. 5), and a matching circuit, if necessary. An extra UWB sensor may also be incorporated, in certain embodiments, for calibration purposes. In various embodiments, the design is configured to achieve the desired sensitivity of detecting ablation growth (with a resolution of <3 mm and depth of up to 20 mm).

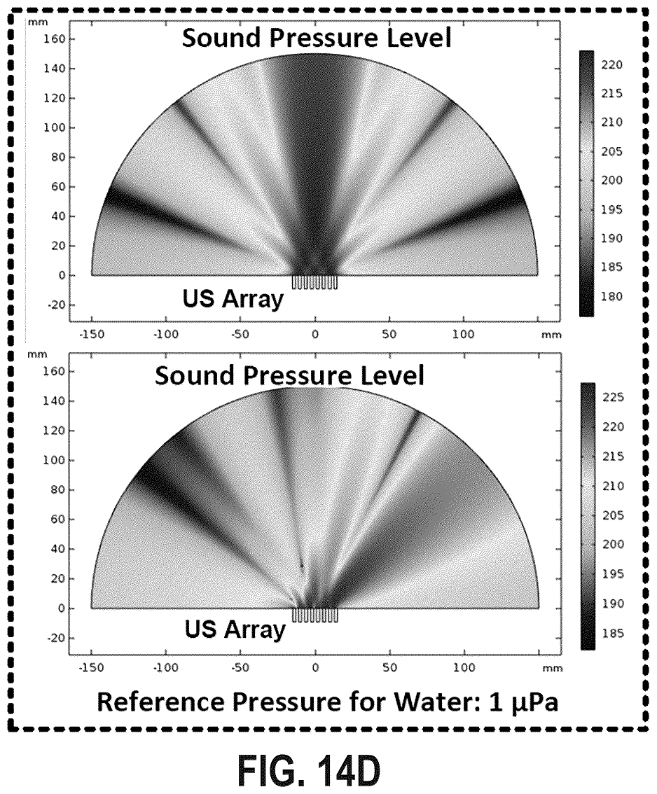

In various embodiments, the UWB sensing antenna 140 may comprise a UWB fractal antenna. Accordingly, a conceptual schematic of an exemplary UWB fractal antenna is shown in FIG. 15. In various embodiments, the UWB fractal antenna is designed to operate within the UWB frequency range, 3-12 GHZ, to (1) detect changes in the spatial position and size of thermally ablated brain tissue based on variations in the reflection coefficient (S₁₁), and (2) support real-time thermal ablation feedback to minimize damage to surrounding healthy neural structures.

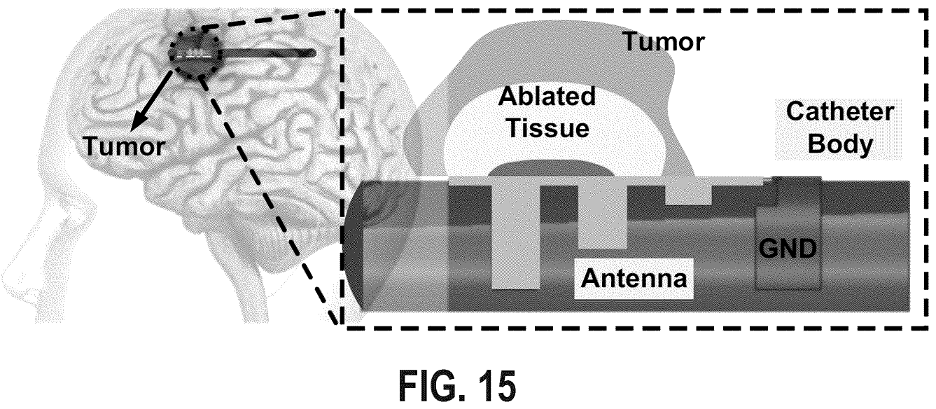

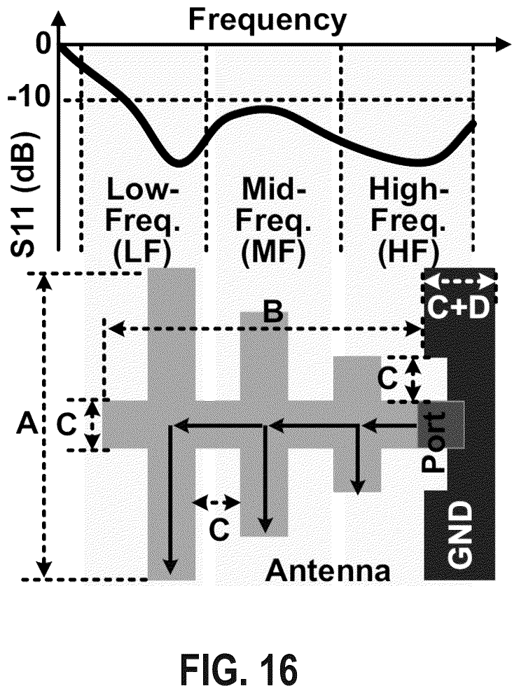

In general, a UWB fractal antenna is a compact, high-performance antenna that uses self-similar, repeating geometric shapes to operate efficiently over an extremely wide frequency range (typically 3.1 GHz to 10.6 GHZ). By using fractal patterns like rectangles, the antennas maximize the edge length within a small area, offering miniaturization, multi-band capabilities, sensitivity to variations in the surrounding loading medium, and high gain for consumer electronics, IoT, and medical imaging. The UWB fractal antenna shown in FIG. 16 is designed with a compact symmetric geometry, where when the length of the antenna increases, it becomes more efficient in lower frequencies relatively. The antenna is divided into three regions: low-frequency (LF), mid-frequency (MF), and high-frequency (HF). As shown in FIG. 16, the HF region is located closest to the feed port, while the LF region is positioned farthest from the port. When burn tissue variation occurs at different parts of the antenna, corresponding changes are observed in the reflection coefficient (S11) within the related frequency range. This behavior is conceptually illustrated in FIG. 17, where variations in different antenna regions affect the associated LF, MF, or HF portions of the UWB frequency band.

In various embodiments, the UWB fractal antenna is adapted to the cylindrical shape and size of a catheter with 1.5 mm radius, as shown in FIG. 17. It supports wideband operation across the UWB frequency range. FIG. 17 also presents the detailed antenna geometry, including the middle view of the fractal copper structure with labeled dimensional parameters (A-H), as well as the front view illustrating the antenna with an insulation layer applied.

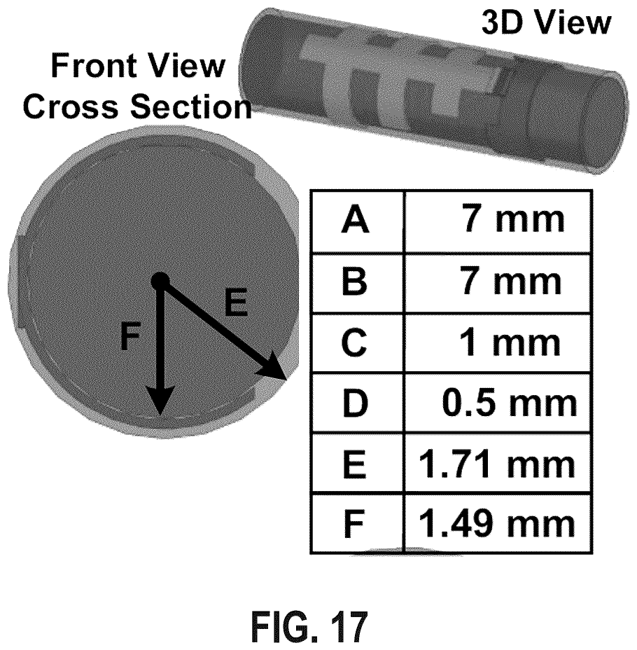

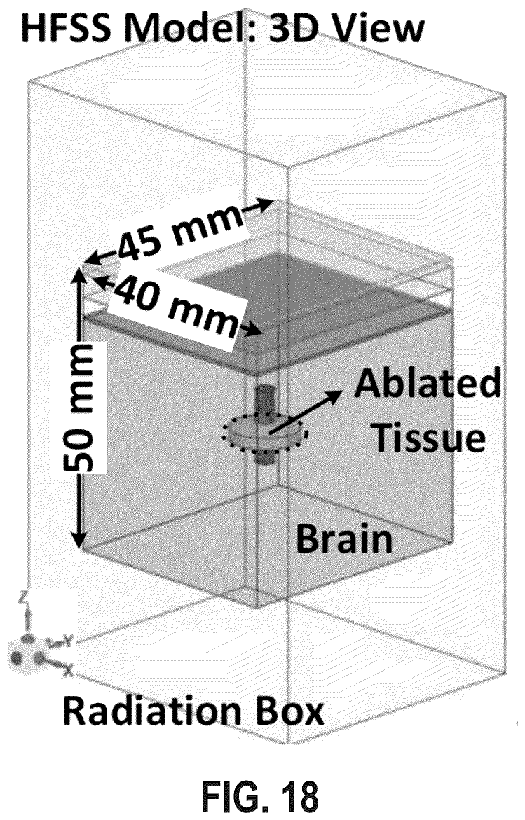

The disclosed antenna system is modeled using ANSYS HFSS, and its performance is evaluated within a brain tissue model, shown in FIG. 18. The simulation model represents a biologically realistic human head composed of six concentric tissue layers: skin, fat, bone, dura, cerebrospinal fluid (CSF), and brain. A burn tissue zone is embedded within the brain layer to mimic localized ablation. The entire structure is enclosed within a 100 mm×60 mm×60 mm radiation box to replicate free-space boundary conditions. Side and front views clearly show the spatial configuration of the tissue model and the embedded single-antenna system, shown in FIG. 19.

A burn region is embedded within the brain layer, and its location and size are systematically varied to study the antenna's electromagnetic response across the operating frequency band. The electromagnetic simulation of the disclosed system was carried out using Ansys HFSS, a full-wave 3D electromagnetic solver widely used in high-frequency biomedical modeling. The objective was to evaluate the sensitivity of the single UWB fractal antenna to variations in the surrounding biological environment by analyzing the reflection coefficient t (S₁₁) over the 2-c multilayer head model was constructed, containing concentric tissue layers. Each layer was assigned appropriate dielectric properties and thicknesses based on biomedical literature. A burn tissue region was embedded within the brain layer and systematically varied in both vertical location and size to simulate different ablation scenarios.

The effect of ablated tissue location was investigated by positioning the ablated region at three axial levels relative to the antenna structure (LF, MF, and HF), corresponding to zones of different field concentration. Additionally, the influence of burn tissue size/growth was evaluated by comparing small and large volumetric targets at fixed positions. These studies aim to determine how spatial and geometric changes in the tissue affect the antenna's electromagnetic response, particularly the S₁₁ performance, which is critical for detecting ablation depth and position.

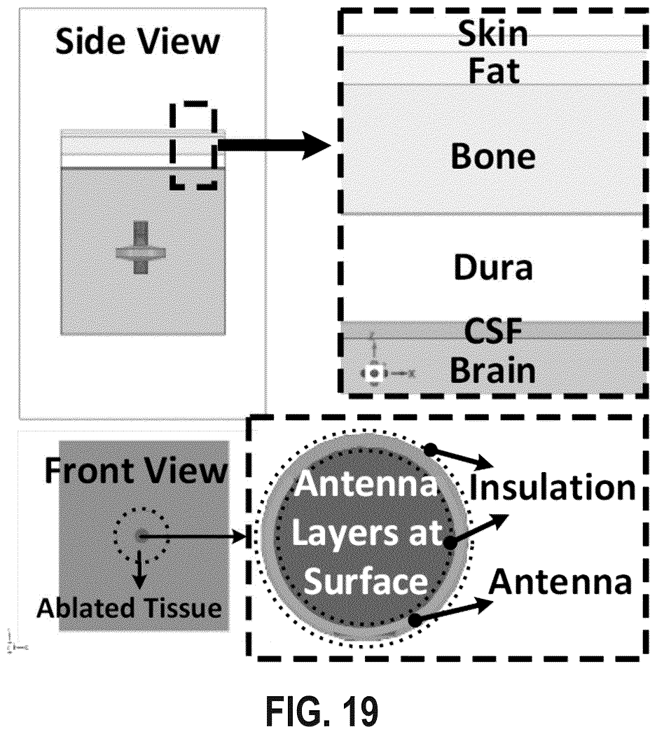

To assess the impact of tissue characteristics on antenna performance, the position and volume of the ablation zone were varied, as shown in FIGS. 20, 21, and 22, representing positional and volumetric variations, respectively. These configurations were designed to examine the frequency-dependent response of the antenna to changes in the ablation zone. It is verified that the UWB antennas have a significant impact on antenna performance. In particular, FIG. 20 illustrates the simulated S₁₁ response for different tissue locations across the 2-12 GHz frequency window. The antenna exhibits high sensitivity to different positions, with each configuration resulting in noticeably different reflection characteristics. For ablated tissue placed at Location 1, the antenna shows strong resonance behavior with deep S₁₁ notches near 5.6 GHz, achieving a minimum return loss of −27 dB. This configuration demonstrates optimal impedance matching and high absorption efficiency due to its proximity to the lower resonator elements. When the ablation happens at Location 2, the S₁₁ profile shifts leftward in frequency, and the depth of resonance increases due to its higher frequency changes that happen in frequencies above 12 GHZ (dual resonance, bifurcation range is getting wider relatively). The minimum S₁₁ is approximately-40 dB, with broader bandwidth observed around 5 GHz. At Location 3, closest to the top of the antenna, the reflection response becomes shallower and less defined. The resonance peak shifts further to the left (other branch of the bifurcation is moved to higher frequencies, larger bifurcation is experienced), and the minimum S₁₁ is reduced to −18.2 dB around 4.7 GHZ. Overall, When the ablated tissue is positioned closer to the feed port, the S₁₁ resonance exhibits a noticeable shift toward lower frequencies (higher bifurcation level, resonance at higher frequency is shifted at a higher frequency). This behavior confirms that the antenna exhibits strong location-dependent electromagnetic characteristics.

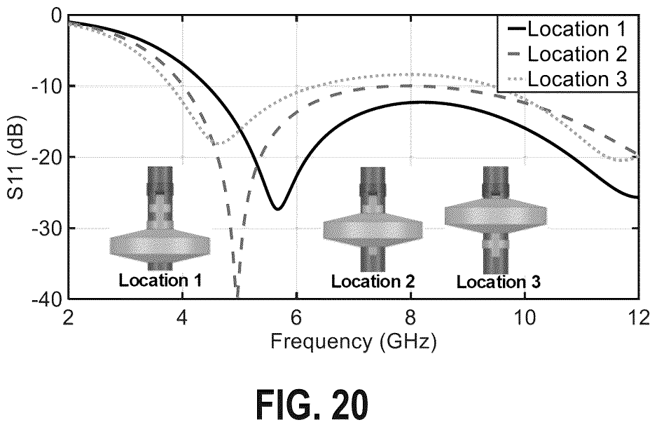

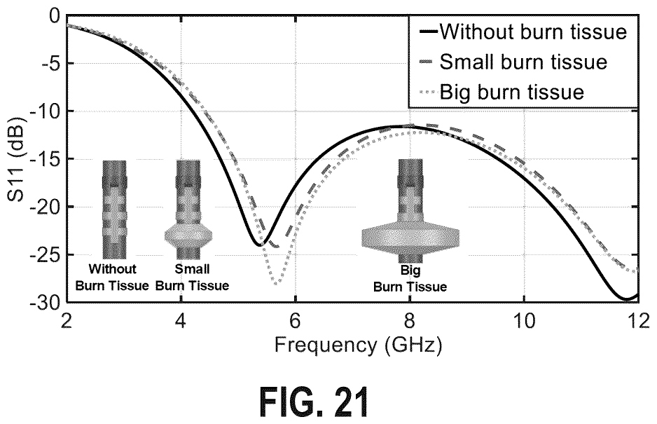

To evaluate the effect of ablated tissue volume on antenna performance, two burn tissue sizes (small and large) were introduced at the low-frequency (LF) and high-frequency (HF) sections of the antenna structure, as illustrated in FIGS. 21 and 22. This configuration enables studying how ablation size influences the antenna reflection characteristics.

As shown in FIG. 21, when the ablated tissue is located in the LF section of the antenna, the larger ablation volume produces a deeper resonance at 5.6 GHz, with a minimum S₁₁ value of −27 dB. In contrast, the smaller ablated tissue slightly shifts the resonance to 5.7 GHZ, accompanied by a shallower minimum of approximately −24 dB. In the HF region, the resonance frequency and S₁₁ magnitude remain unchanged for both ablated tissue sizes. Overall, variations in ablated tissue size within the LF section result in an observable 0.1 GHz frequency shift and an approximately 3 dB difference in S₁₁ magnitude, indicating that changes in ablated tissue volume near the LF section significantly affect low-frequency resonance behavior.

As illustrated in FIG. 22, variations in burn tissue size located in the HF section of the antenna have a pronounced impact on the high-frequency S₁₁ response. Specifically, the smaller burn tissue volume produces a deeper resonance at 11.1 GHZ, with a minimum S₁₁ value of −29 dB, whereas the larger ablation volume shifts the resonance to 8.9 GHZ with a shallower minimum of approximately-26 dB. This corresponds to a 2.2 GHz frequency shift and an approximately 3 dB difference in resonance depth in the high-frequency region. In contrast, the low-frequency S₁₁ response exhibits relatively minor changes, with only a 1.7 dB variation in resonance depth and an approximate 1 GHz frequency shift.

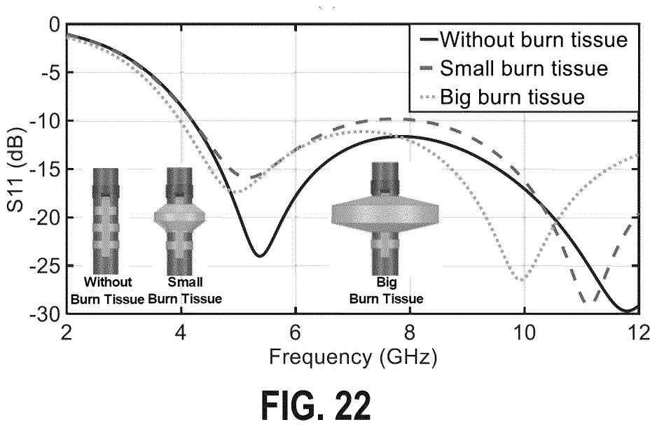

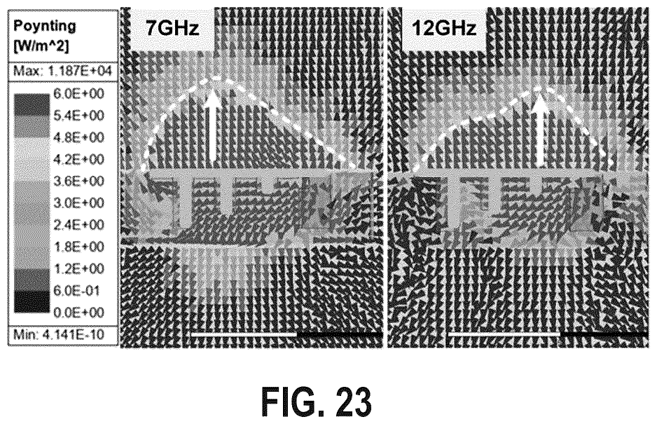

A cut plane was defined through the center of the antenna to evaluate the Poynting vector distribution at both low and high frequencies. The results demonstrate that electromagnetic power flow exhibits distinct spatial characteristics at different frequency bands, where the Poynting vector simulation of fractal antenna verifies the dependency of wave propagation location to the frequency, as shown in FIG. 23. This simulation explains why the antenna is sensitive to the location variations of phase changes of tissue. To avoid damaging healthy brain tissue beyond the tumor boundary, where exposed by UWB electromagnetic fields, we have calculated the specific absorption rate (SAR) in brain tissue by the HFSS software using the model of FIG. 3(a). We have set the power input of the antenna at 2 mW, which is significant enough to detect changes of up to 2 cm. As shown in FIG. 24, the calculated average SAR remains below 1.6 W/kg, confirming that the disclosed fractal UWB antenna operates within safe limits for biomedical treatment applications.

The disclosed UWB fractal antenna demonstrates a strong sensitivity to both the spatial location and volume variation of thermally ablated brain tumor. When the ablated tissue is positioned at different surface locations along the antenna structure, the corresponding resonant frequency bands exhibit selective responses. Specifically, as the ablated tissue moves closer to the feed port, the S11 resonance undergoes a distinct shift and enlarged bifurcation level. These results are visualized by calculation the Poynting vector, as well. In addition, variations in ablated tissue volume further modulate the antenna reflection characteristics. Larger tissue volumes induce more pronounced frequency shifts (up to 2.2 GHZ) and resonance depth variations (3 dB) within the associated frequency bands. Additionally, the safety of the UWB fractal antenna sensor when healthy tissues are exposed to its electromagnetic fields has been demonstrated by calculating the SAR, resulting in a value below the standard of 1.6 W/kg. The present disclosure confirms the location- and volume-dependent frequency response along the antenna structure, validating the disclosed antenna's capability for real-time monitoring of ablation progression and supporting its potential application in closed-loop, precision, and adaptive thermal therapy.

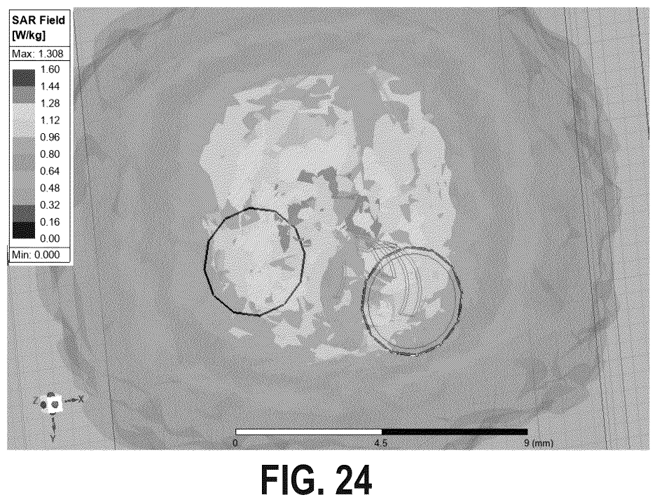

Next, an illustrative and non-limiting assembly process is depicted in FIG. 25, where a 3D printed frame is utilized at the catheter tip to properly position the components from steps 1 to 4, which correspond to assembling of a heat source (Peltier module) 120, assembly of ultrasonic transducer array 130, assembly of UWB sensor 140, and assembly of MRI positioning coil 150 in the tip of the TPA catheter apparatus 110. The components and ultrasound transducer elements pitches are configured to achieve optimal heat control and accurate feedback, while the MRI positioning coil is provided at the catheter tip to enable MRI navigation. In various embodiments, coaxial and regular cables (AWG 26-48) connect the catheter tip to the interface circuits (<40 cm). In various embodiments, the catheter body is made of biocompatible materials, and the catheter tip is be covered with biocompatible materials (with high-temperature tolerance) to prevent moisture-induced short circuits. For a prototype of the catheter 110 and subsequent testing, Polyimide (Kapton) Tape is used to cover the catheter tip, offering excellent temperature tolerance and biocompatibility with efficient heat conductivity.

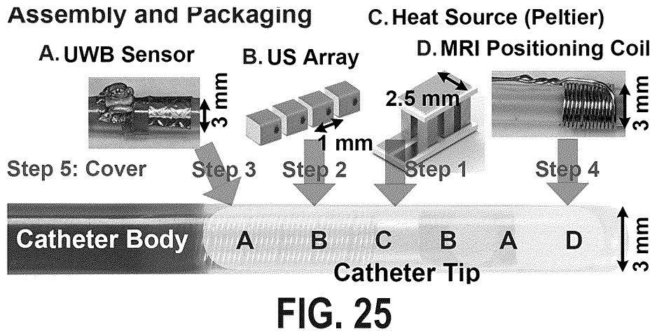

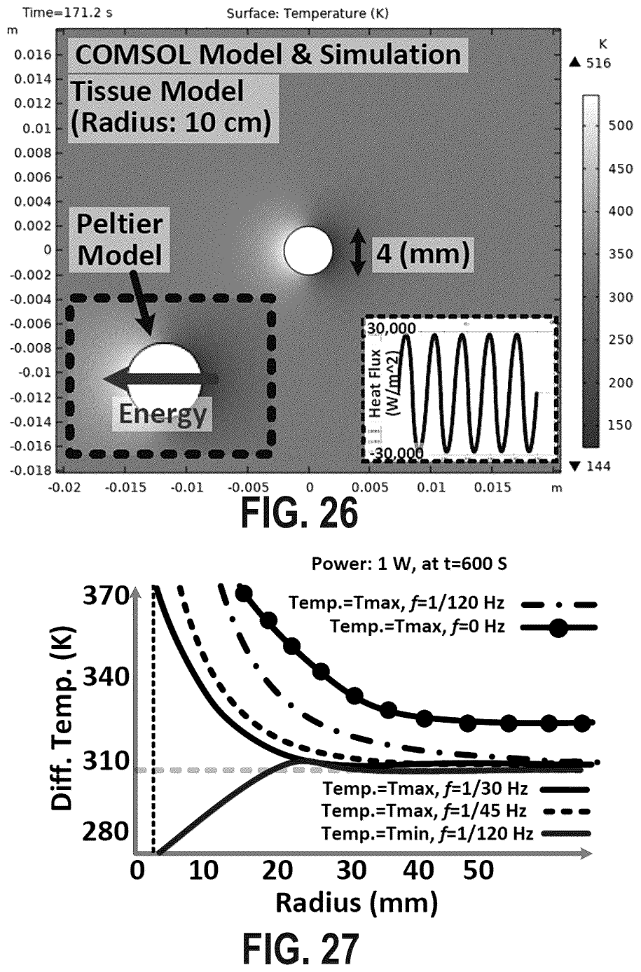

For analysis purposes, COMSOL Multiphysics software has been used to model aspects of the TPA catheter system. In particular, the software has been used to a model the Peltier module and simulate heat transfer in a 2D tissue model. FIG. 26 displays the COMSOL model (Peltier module radius of 2 mm and solving domain radius of 20 cm) and the simulation results of temperature changes, considering an applied heat flux (30,000 W/m², ~1 W) with a frequency of 1/120 Hz and a total time of 600s. FIG. 27 presents the simulation results of maximum or minimum achieved temperatures at different radii, comparing the conventional approach (constant heat flux; f=0) with the exemplary system at various frequencies of 1/120, 1/45, and 1/30 (Hz). The results show that the TPA catheter system can limit the heated area with a near-zero temperature gradient, allowing it to settle at the body's original temperature.

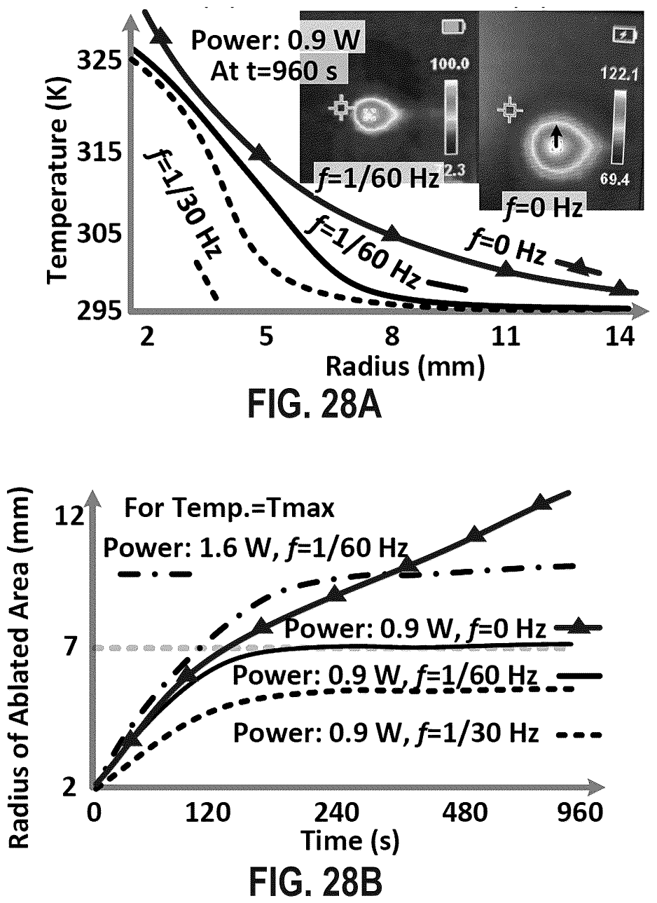

To assess the feasibility of the exemplary TPA catheter system 100, two catheter prototypes are implemented for in vitro experiments. Kapton tapes are used for electrical and moisture isolation. Temperature measurements are performed using a thermal imager (Klein Tools TI250). The test setup includes fresh beef meat, with the TPA catheter inserted 1 cm into the meat. The ablated area experiences temperature changes up to 30° C. A comparison is made of the measured temperatures and the conventional (constant heat; f=0) and the exemplary (f=1/60 Hz) systems. The measured results, summarized in FIGS. 28A and 28B, indicate the superiority of the disclosed design in limiting heat penetration depth. FIG. 28B shows the temperature as a function of radius (at t=960 s) for different frequencies, demonstrating that reducing the frequency increases the ablation area, while f=0 does not settle down to room temperature, 295K. FIG. 28B shows the radius of the ablated area as a function of time for different power levels and frequencies, indicating for non-zero frequencies, the thermal boundary of the ablated areas is limited after around 200 s.

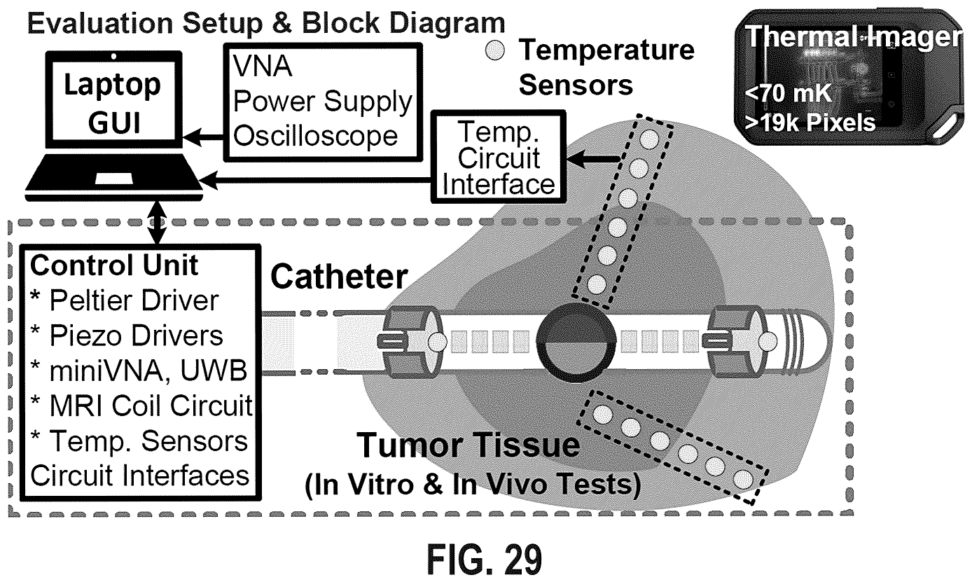

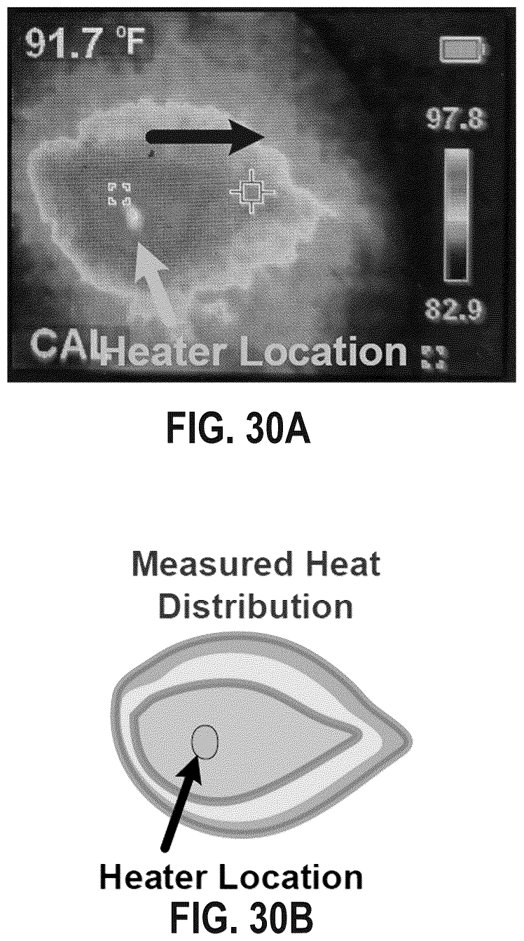

To assess the impact of ultrasound waves on heat directing, a test setup comprised of a piezoelectric ultrasound transducer, chicken breast, a mm-sized Peltier-based heat source, and a thermal imager, was used to show the results of heat propagation directed towards the ultrasound direction (left to right), as depicted in FIG. 29. Further, FIG. 30B depicts a location of the heat source in relation to the measured heat distribution, and FIG. 30A demonstrates the thermal image showing the heat propagation, confirming the effectiveness of the disclosed system in directing heat via ultrasound waves. A similar test was done with no ultrasound and the distribution of heat was near isotropic.

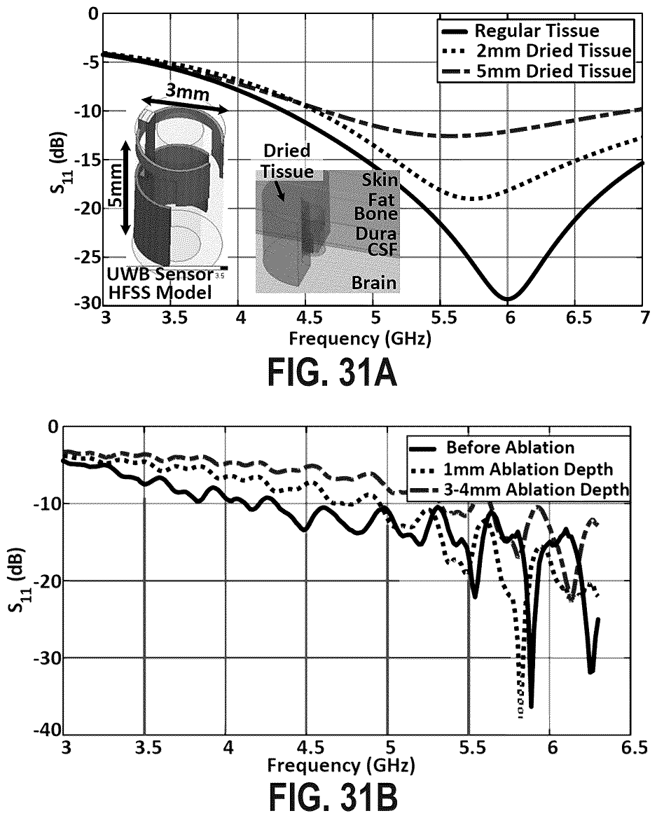

As previously mentioned, ANSYS HFSS software was used to model the UWB sensor 140 to detect tissue property changes in the antenna's near field (3 GHZ-12 GHZ). Realistic human tissue models were employed in the simulation. FIG. 31A shows simulations results of the HFSS model and the UWB sensor and human tissue layers (skin, fat, bone, dura, CSF, and brain). The simulation results demonstrate the reflection coefficient (S11) as a function of frequency for different scenarios, revealing stronger reflections (higher S11) after ablation (dried tissue models) with varying thickness. Further, to assess the UWB sensor's ability to differentiate tissue before and after ablation, a cylindrical UWB sensor was implemented using copper foil covered with Kapton tape material. FIG. 31B shows the measured results which demonstrate the UWB sensor's sensitivity to changes in tissue properties (S11 vs. frequency).

In summary, the present disclosure presents a novel concept of an exemplary Tumor Precision Ablation (TPA) catheter system that is configured to direct heat in different directions to enable precise ablation of unsymmetrical tumors by affecting heat transfer with ultrasound waves. By utilizing ultrasound waves, which are non-heat producing and controllable via beamforming using arrays of ultrasound transducers, heat flux can be directed in desired directions. As such, ultrasound waves can direct the heat, leveraging the phonon phenomenon, and creating a heat-guide channel with higher thermal conductivity. Also, by alternating between heating and freezing phases, the ablation thermal boundary can be controlled, resulting in a temperature gradient reduction at the tumor edge. Further, UWB sensors (e.g., mm-sized UWB sensors) can detect changes in the dielectric property of tissue during ablation and estimate the volume of ablated tissue.

The novel Peltier-based catheter (PBC) demonstrated through COMSOL Multiphysics simulations offers a precise and controlled method for brain tumor ablation. Unlike conventional techniques, the disclosed PBC successfully confines thermal energy within tumor boundaries, significantly reducing the risk of collateral damage to healthy tissues. By adjusting the AC input frequency, the PBC effectively ablates the tumors of various sizes. The present disclosure specifically ablates the tumors within a range of approximately 3.5 mm to 8 mm. The maintenance of a near-zero temperature gradient at tumor edges highlights the catheter's precision and its potential to minimize tumor recurrence rates. Overall, this innovative catheter design presents a promising advancement in thermal ablation therapies, providing safer, more targeted, and clinically effective treatment outcomes.

In various embodiments, an exemplary method of the present disclosure involves positioning a catheter in a portion of tissue during an ablation, where the catheter comprises a heat source, one or more ultrasound (US) transducer arrays, an ultra-wideband (UWB) sensor, and a control unit for controlling individual components of the catheter; generating, by the heat source, a transfer of heat, wherein the heat source alternates heating and freezing cycles at different frequencies to achieve a near zero-temperature gradient at a tumor boundary; directing, by beamforming, the transfer of heat in a desired direction using ultrasound waves via the one or more US transducer arrays; and/or detecting, by the UWB sensor, changes in tumor properties during ablation.

In various embodiments, an exemplary catheter further comprises a magnetic resonance imaging (MRI) coil and the method further comprising tracking, using the MRI coil, a positioning of the catheter. In various embodiments, the transfer of heat is directed by applying different pulse patterns to control heat penetration depth. In various embodiments, the heating and freezing cycles are alternated at different frequencies to set a clinically defined radius for the heat penetration depth. In various embodiments, the heat source comprises one or more Peltier modules that produce heating and freezing effects. In various embodiments, the ultrasound transducer array comprises a 1D array. In various embodiments, the 1D array of ultrasound transducers comprises 4 to 8 transducer piezo elements. In various embodiments, the one or more US transducer arrays comprise two US transducer arrays are positioned adjacent to the heat source in the catheter, enabling heat being directed in forward and backward directions.

In various embodiments, an exemplary method further comprises rotating the catheter during an ablation procedure to cover an entire tumor. In various embodiments, the one or more US transducer arrays comprise a single US transducer array having 2 US transducer elements tilted at +45°. In various embodiments, the UWB sensor detects dielectric measurements by measuring absorption and reflection of UWB signals in body tissues. In various embodiments, the USB sensor operates in a frequency range of 3 GHZ to 12 GHz.

The present disclosure offers significant benefits to society by addressing the risk of ablating healthy tissues, saving the lives of millions of patients with tumors. The number of cancer cases in North America was >1.8 million in 2020, with an average cost of $5 k-$10 k per case. With an estimated growth rate of over 13.6%, the global tumor ablation market size will be more than $3.8B in 2030. This research aims to improve patient outcomes and reduce healthcare burdens. This has the potential to revolutionize the treatment landscape by providing a risk-mitigated precision ablation option, benefiting individuals. The disclosed technology can be used for the ablation of brain, liver, lung, kidney, and bone tumors, treating epilepsy, treating brain vascular abnormalities, lung denervation, and atrial fibrillation ablation, offering safer and more effective treatment options. By deepening our understanding of how low-power ultrasound waves interact with heat distribution, the present disclosure can contribute to scientific knowledge in a highly specialized field to minimize the risk of ablating healthy tissues during ablation.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y.’”

It should be emphasized that the above-described embodiments of the present invention are merely possible examples of implementations, merely set forth for a clear understanding of the principles of the disclosure. Many variations and modifications may be made to the above-described embodiment(s) without departing substantially from the principles of the present disclosure. All such modifications and variations are intended to be included herein within the scope of this disclosure.

1. A method comprising:
positioning a medical apparatus in or on a portion of tissue during an ablation, where the medical apparatus comprises a heat source and a control unit for controlling individual components of the medical apparatus; and
generating, by the heat source, a transfer of heat, wherein the heat source alternates heating and freezing cycles at different frequencies to achieve a near zero-temperature gradient at a tumor boundary.

2. The method of claim 1, wherein the medical apparatus comprises a magnetic resonance imaging (MRI) coil, the method further comprising tracking, using the MRI coil, a positioning of the medical apparatus.

3. The method of claim 1, wherein the transfer of heat is directed by applying different pulse patterns to control heat penetration depth.

4. The method of claim 3, wherein the heating and freezing cycles are alternated at different frequencies to set a clinically defined radius for the heat penetration depth.

5. The method of claim 1, wherein the heat source comprises one or more Peltier modules that produce heating and freezing effects.

6. The method of claim 1, further comprising rotating the medical apparatus during an ablation procedure to cover an entire tumor.

7. The method of claim 1, wherein the medical apparatus further comprises one or more temperature sensors for detecting the temperature output by the medical apparatus.

8. The method of claim 7, wherein the control unit receives sensor data from the one or more temperature sensors and provides signals for setting ablation phase and duration parameters.

9. The method of claim 7, wherein the different frequencies include 1/120 Hz, 1/60 Hz, 1/45 Hz, and 1/30 Hz.

10. The method of claim 7, wherein heat distribution is inversely related to an input frequency of the heat source.

11. A system comprising:
a Tumor Precision Ablation (TPA) medical apparatus having a heat source and a control unit for controlling individual components of the TPA medical apparatus,
wherein the heat source is configured to alternate heating and freezing cycles at different frequencies to achieve a near zero-temperature gradient at a tumor boundary of a subject.

12. The system of claim 11, wherein the TPA medical apparatus comprises a magnetic resonance imaging (MRI) coil.

13. The system of claim 11, wherein the heat source is configured to apply different pulse patterns to control heat penetration depth.

14. The system of claim 13, wherein the heating and freezing cycles are alternated at different frequencies to set a clinically defined radius for the heat penetration depth.

15. The system of claim 11, wherein the heat source comprises one or more Peltier modules that produce heating and freezing effects.

16. The system of claim 11, wherein the TPA medical apparatus further comprises one or more temperature sensors for detecting the temperature output by the medical apparatus.

17. The system of claim 16, wherein the control unit is configured to receive sensor data from the one or more temperature sensors and provide signals for setting ablation phase and duration parameters.

18. The system of claim 11, wherein the different frequencies include 1/120 Hz, 1/60 Hz, 1/45 Hz, and 1/30 Hz.

19. The system of claim 11, wherein heat distribution is inversely related to an input frequency of the heat source.

20. The system of claim 11, wherein the TPA medical apparatus comprises a TPA catheter device.
